(19) United States
(12) Patent Application Publication
(10) Pub. No.: US 2025/0269344 A1
(43) Pub. Date: Aug. 28, 2025

(54) PROCESS FOR PREPARING AN ENCAPSULATED COMPOSITION

(71) Applicants: Venkata Ravi Sankar UMMIDI, Ramchandrapuram (IN); Taraka Ramji MOTURU, Brussels (BE)

(72) Inventors: Venkata Ravi Sankar UMMIDI, Ramchandrapuram (IN); Taraka Ramji MOTURU, Brussels (BE)

(21) Appl. No.: 18/589,405

(22) Filed: Feb. 27, 2024

(51) Int. Cl. B01J 13/04

(52) U.S. Cl. CPC B01J 13/043

(57) ABSTRACT

A process for preparing an encapsulated composition is disclosed. The disclosed process uses clay as an encapsulation material. An encapsulated composition obtained using the disclosed process is also disclosed. The disclosed encapsulated composition finds application in the food, agricultural/environmental, cosmetic, and pharmaceutical industries and has been found to be efficient for the formulation of sustained-release pesticides.

### FIELD OF INVENTION

The present disclosure relates to a process for preparing an encapsulated composition. In particular, the present disclosure relates to a process for preparing the encapsulated composition using clay.

### BACKGROUND

Encapsulation of bioactive compounds is known for food and agricultural applications. However, existing methods of achieving encapsulation generally require one or more harsh processing conditions, such as the use of organic solvents and/or high temperatures. This causes degradation or inactivation of the bioactive compounds during the encapsulation process and necessitates high percent loadings of bioactive compounds or the use of expensive polymers and/or processing techniques. Thus, there is a need for improved methods and compositions for preparing encapsulated systems that could exhibit improved stability, a high release time, retain and protect the properties of bioactive compounds, and maintain and present bioactivities even in extreme thermal, radiation, and pH conditions, while being cost effective.

### SUMMARY

A process for preparing an encapsulated composition is disclosed. The disclosed process comprises, first, heating a clay at a temperature in the range of 200 to 250° C. for a time-period of 3-5 hours. The heated clay is mixed with water in a w/w ratio in the range of 1:5-1:10 to obtain a suspension of clay, followed by the separation of the suspension of clay into a sediment of clay and an aqueous layer. The aqueous layer is decanted, followed by the addition of an acid to the clay sediment to obtain purified clay. In the next step, an aqueous slurry of the purified clay is prepared. The pH of the aqueous slurry of the purified clay is adjusted in the range of 9-11. Further, at least one active ingredient is blended with the aqueous slurry of the purified clay to obtain a mixture of the active ingredient and the purified clay. The mixture of the active ingredient and the purified clay is subjected to drying so that the encapsulated composition, comprising the active ingredient homogenized with the clay, is obtained.

An encapsulated composition obtained using the disclosed process is also disclosed. Said encapsulated composition comprises at least one active ingredient homogenised with a clay wherein the active ingredient is selected from the group consisting of essential oils, bio-stimulants, fertilizers, pesticides, and any blend of natural ingredients.

### DETAILED DESCRIPTION

To promote an understanding of the principles of the disclosure, reference will now be made to embodiments and specific language will be used to describe the same. It will nevertheless be understood that no limitation of the scope of the disclosure is thereby intended, such alterations and further modifications in the disclosed composition and process, and such further applications of the principles of the disclosure therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “one embodiment” “an embodiment” or similar language means that a particular feature, structure, or characteristic described in connection with the embodiment is included in at least one embodiment of the present disclosure. Thus, appearances of the phrase “in one embodiment”, “in an embodiment” and similar language throughout this specification may, but do not necessarily, all refer to the same embodiment.

Essential oils are concentrated hydrophobic liquids containing volatile compounds obtained from the fruit, seeds, flowers, bark, stems, roots, leaves or other parts of plants. The term “essential oil” as used herein refers to essential oil as a whole or the specific compound(s) extracted from the essential oil.

In an aspect, the present disclosure relates to a process for preparing an encapsulated composition. Said process comprises the step of:
a. heating a clay at a temperature in a range of 200 to 250° C. for a time period in a range of 3-5 hours;
b. mixing the heated clay with water in a w/w ratio in a range of 1:5-1:10 to obtain a suspension of clay, followed by separation of suspension of clay into a sediment of clay and an aqueous layer;
c. decanting the aqueous layer, followed by the addition of an acid to the sediment of clay to obtain purified clay;
d. preparing an aqueous slurry of the purified clay;
e. adjusting the pH of the aqueous slurry of the purified clay in the range of 9-11;
f. blending an active ingredient with the aqueous slurry of the purified clay to obtain a mixture of the active ingredient and the purified clay; and
g. subjecting the mixture of the active ingredient and the purified clay to drying such that the encapsulated composition comprising of the active ingredient homogenised with the clay is obtained.

In the disclosed process, the purified clay interacts with the active ingredients to form a network of non-covalent bonds (hydrogen bonds), such that the active ingredient is encapsulated within the matrix of the purified clay. This provides for slow and sustained release of the active ingredients from the composition. Thus, the disclosed process causes effective encapsulation of the active ingredient while avoiding any harsh process conditions and/or raw materials which could degrade the bioactivity of the active ingredient.

In another aspect, an encapsulated composition obtained using the disclosed process is disclosed. Said encapsulated composition comprises an active ingredient homogenised with a clay wherein the active ingredient is selected from the group consisting of essential oils, bio-stimulants, fertilizers, pesticides, and any blend of natural ingredients.

In an embodiment, the clay is selected from the group consisting of montmorillonite (MMT), bentonite clay, kaolinite, dickite, amesite, lizardite, smectite, palygorskite, sepiolite, glauconite, mica, vermiculite, saponite halloysite, hectorite, and combinations thereof. In some embodiments, the clay is bentonite clay. The bentonite clay includes one or more of sodium bentonite, and calcium bentonite clay. In an embodiment, the bentonite clay has an average particle size in the range of 1-10 μm. In some embodiments, the bentonite clay has the average particle size in the range of 7 μm. In an embodiment, the bentonite clay has a mass density in the range of 1.2-1.8 g/cm³. In some embodiments, the bentonite clay has the mass density in the range of 1.5 g/cm³. In an embodiment, the bentonite clay has a cation exchange capacity (CEC) in the range of 60 to 150 meq/100 g. In some embodiments, the bentonite clay has the cation exchange capacity (CEC) in the range of 120 meq/100 g.

In the first step of obtaining purified clay, the clay is heated to a predetermined temperature. The clay is heated using a method known in the art. In an embodiment, the clay is heated in an oven at 250° C. for 4 hours. In an embodiment, after heating, the heated clay and water are mixed at a temperature ranging between 60-80° C. under continuous stirring at a stirring rate of 2000 to 4000 rpm to obtain the suspension of clay. The obtained suspension of clay is allowed to settle for a time-period in a range of 120 to 180 minutes to separate into the sediment of clay and the aqueous layer. After settling of the suspension, the acid is added to the sediment of clay at a temperature in a range of 25-30° C. under continuous stirring to obtain the purified clay. In an embodiment, the acid is selected from the group consisting of sulphuric acid, hydrochloric acid and a combination thereof. In some embodiments, the acid is hydrochloric acid having molarity in the range of 3-7M.

After purification, the purified clay is used to prepare the aqueous slurry of the purified clay. In an embodiment, the purified clay is mixed with water in a w/w ratio ranging from 1:2 to 1:5 under continuous stirring to obtain the aqueous slurry of the purified clay. In an embodiment, the stirring is done at a rate ranging between 200 to 4000 rpm for a time period in a range of 60-120 min. In some embodiments, the aqueous slurry of the purified clay is stirred at 3000 rpm for 60 minutes.

In the next step, pH of the aqueous slurry of the purified clay is adjusted by adding a base. In an embodiment, the base is selected from the group consisting of sodium hydroxide, potassium hydroxide, calcium hydroxide, and their combinations. In some embodiments, the base is sodium hydroxide. In some embodiments, the pH of the aqueous slurry of the purified clay is adjusted to 10.

In the next step, the active ingredient is blended with the aqueous slurry of the purified clay to obtain the mixture of the active ingredient and the purified clay. In an embodiment, the active ingredient is blended with the aqueous slurry of the purified clay in a w/w ratio in the range of 1:5 to 2:5. In some embodiments, the active ingredient is blended with the aqueous slurry of the purified slurry in the w/w ratio of 0.01:1. In an embodiment, the active ingredient is blended with the aqueous slurry of the purified clay at a temperature in a range of 60 C under continuous stirring. In some embodiments, the active ingredient is blended with the aqueous slurry of the clay at 70° C. with stirring at a stirring rate of 3000 rpm.

In an embodiment, the active ingredient is mixed with a solvent prior to blending with the aqueous slurry of the purified clay. The addition of the solvent to the active ingredient assists in the adsorption thereof into the pores of the purified clay. In an embodiment, the solvent is selected from the group consisting of ethanol, isopropanol, butanol and combinations thereof. In some embodiments, the solvent is ethanol.

In an embodiment, after the addition of the active ingredient, the aqueous slurry of the purified clay is stirred at a stirring rate of 3000 rpm for a time-period in the range of 60 min.

In the next step, the obtained mixture of the active ingredient and the purified clay is subjected to drying using a drying technique now known or in the future developed. Examples of drying method include but are not limited to spray drying, hot air oven drying or tray drying. In some embodiments, the obtained aqueous slurry of the clay is dried in a hot air oven. In an embodiment, the obtained mixture of the active ingredient and the purified clay is dried in the oven with air circulation at a temperature in the range of 50-55° C. such that the encapsulated composition comprising the active ingredient homogenized with the purified clay is obtained.

In an embodiment, homogenization of purified clay with the active ingredient causes encapsulation of the active ingredient with the clay. In an embodiment, the encapsulation includes microencapsulation to form microspheres comprising the active ingredient encapsulated with the purified clay. In an embodiment, the microspheres of the active ingredient have an average particle size (D₅₀) in the range of 0.5 to 5 μm. In some embodiments, the microspheres have the average particle size (D50) of 3 μm. In an embodiment, the microspheres has a tapped density in a range of 1.016 g/cm³-1.035 g/cm³.

In an embodiment, the active ingredient is selected from the group consisting of essential oils, bio-stimulants, fertilizers, pesticides, and any blend of natural ingredients. In an embodiment, the active ingredient is one or more essential oils or an extract thereof. In an embodiment, the one or essential oil includes but is not limited to angelica oil, anise oil, basil oil, bay oil, bergamot oil, bois de rose oil, calendula oil, cananga oil, caraway oil, cardamom oil, cedar oil, cedarwood oil, chamaecyparis obtusa oil, chamomile oil, cinnamon oil, citronella oil, clary sage oil, clove oil, copaiba balsam oil, coriander oil, cumin oil, dill oil, eucalyptus oil, fennel oil, garlic oil, geranium oil, ginger oil, grapefruit oil, guaiacwood oil, hiba oil, camphor oil, iris oil, Japanese mint oil, jasmine oil, lavender oil, laurel leaf oil, lemon oil, lemongrass oil, lime oil, linaloe oil, lindera oil, mandarin oil, mustard oil, neroli oil, onion oil, orange oil, oregano oil, palmarosa oil, parsley oil, patchouli oil, peach kernel oil, pennyroyal oil, pepper oil, peppermint oil, perilla oil, Peru balsam oil, petitgrain oil, pine needle oil, rose oil, rosemary oil, sandalwood oil, spearmint oil, star anis oil, tagetes oil, tea tree oil, tea seed oil, thyme oil, tolu balsam oil, tuberose oil, meric oil, vetivert oil, western mint oil, white micromeria oil, wintergreen oil or any combination thereof.

In an embodiment, the extract of the one or more essential oil includes one or more class of compounds selected from the group consisting of aromatic compounds, terpenes and terpenoids, aldehydes, esters, and their derivatives. In an embodiment, aromatic compounds include phenols, and phenylpropenes. In an embodiment, phenylpropenes and their derivatives include anethole, myristicin, estragole, apiole, cinnamaldehyde, safrole and cuminal. In an embodiment, phenols and their derivatives include cardonol triene, carvacrol, thymol, cardanol acetate, eugenol, and isoeugenol. In an embodiment, terpenes include acyclic, monocyclic, bicyclic, and tricyclic monoterpenes as well as acyclic, monocyclic, and bicyclic sesquiterpenes. The terpenes and terpenoids include menthone, d-limonene, ocimene, sabinene, 3-carene, linalool, (-) trans-caryophyllene, citral, α-phellandrene, terpinolene, eucalyptol, thujone, camphene, α-pinene, β-pinene, nerol, menthol, β-damascenone, geraniol, myrcene, citronellal, citronellol, tricyclene, fenchone, farnesene, chamazulene, nerolidol, farnisol, germacrene d, bisabolene, selinene, patchoulene, valencene, cyperol, eudesmol, camphor, nootkatone, aromadederene, aromadederene oxide, borneol, carvone, bisabolol.

The term ‘bio-stimulant’ is any natural or synthetic substance which when applied to plants enhances nutrition efficiency, abiotic stress tolerance and/or crop quality. In an embodiment, the active ingredient is a bio-stimulant. Examples of the bio-stimulant include but is not limited to seaweed extracts, amino acids, fulvic acid, humic acid, protein hydrolysates, chitosan, plant growth hormones such as cytokinins, auxins, gibberellins, ethylene, absisic acid, and beneficial microbes. In an embodiment, the beneficial microbes but include are not limited to Fenneliformis spp., Gibberella spp., Gigaspora spp., Glomus spp., Laccaria spp., Paraglomus spp., Pisolithus spp., Rhizoglomus spp., Rhizophagus spp., Rhizopogon spp., Scleroderma spp., Suillus spp., Azospirillum spp., Azotobacter spp., Bradyrhizobium spp., Burkholderia spp., Geobacillus spp., Gluconacetobacter spp., Herbaspirillum spp., Lactobacillus spp., Lactococcus spp., Mesorhizobium spp., Mycobacterium spp., Paenibacillus spp., Pantoea spp., Pseudomonas spp., Rhizobium spp., and Rhodopseudomonas spp.

In an embodiment, the active ingredient is a fertilizer. In an embodiment, the fertilizer is selected from the group consisting of nitrogen fertilizers, phosphorus fertilizers, potassium fertilizers, calcium fertilizers, and magnesium fertilizers. Examples of nitrogen fertilizer include but are not limited to ammonium sulphate, urea, ammonium nitrate, calcium ammonium nitrate calcium nitrate calcium cynamide, and ammonium sulphate nitrate. Examples of phosphorus fertilizer include but are not limited to diammonium phosphate, monoammonium phosphate, nitrogen, phosphorus and potassium and single super phosphate. Examples of potassium fertilizers include but are not limited to monopotassium phosphate, potassium chloride, potassium hydroxide, potassium nitrate, potassium thiosulphate, and potassium magnesium sulphate. Examples of calcium fertilizer include but are not limited to calcium ammonium nitrate and calcium nitrate. Examples of magnesium fertilizer include but are not limited to magnesium nitrate, sulfate of potash magnesia, epsom salt and dolomite.

The term “pesticide” refers to a molecule or combination of molecules that repels, inhibits or kills insects and/or unwanted plants, and can be used for crop protection. In an embodiment, the pesticide includes but are not limited to insecticides, herbicides. fungicides, and nematicides.

In an embodiment, the insecticide is selected from the group consisting of organochlorides, organophosphates, benzoyl ureas, carbamates, pyrethroids, phenyl amides, butenolides, trazines, and phenylpyrazoles. Examples of organochlorides include but are not limited to dicofol, eldrin, dieldrin, chlorobenziate, lindane, benzene bexachloride, methoxychloro aldrin, chlordane, heptaclor, endosufan, isodrin, isobenzan, toxaphene, and chloropropylate. Examples of organophosphates include but are not limited to dimefox, mipafox, methyl parathion, ronnel, enitrothion, bidrin, phorate, fenthion, caumphos, abate, dichlorovas, diptrex, phosphomidon, demetox, oxydemeton-methyl, malathion, dimethoate, and trichlorofan. Examples of benzoyl ureas include but are not limited to novaluron, lufenuron, chlorfluazuron, flufenoxuron, hexaflumuron, noviflumuron, teflubenzuron, triflumuron and diflubenzuron. Examples of carbamates include but are not limited to aldicarb, carbaryl, propoxur, oxamyl and terbucarb. Examples of pyrethroids include but are not limited to allethrin, bonthrin, dimethrin, tetramethrin, ptrethrin, cyclethrin, furethrin, fenevelerate, alphamethrin, decamethrin, and cypermethrin. Examples of phenyl amides include but are not limited barban, carbetamide, chlororprofan, prophan, phenyl urea, fenuron, monuron, diuron, flumeturon, chloroxuron, neburon, and bromuron. Examples of trazines include but are not limited to atrazine, simazine, ametryn, atratone, chlorazine, cynazine, cyprazine, metribuzin, propazine, turbutryn, and simetryn. Examples of phenylpyrazoles include but are not limited to imidacloprid, acetamiprid, thiacloprid, dinotefuran, thiamethoxam and fipronil.

In an embodiment, the fungicide is selected from the group consisting of conazoles, morpholines, strobilurins, phthalonitriles. Examples of conazoles include but are not limited to epoxiconazole, hexaconazole, propiconazole, prochloraz, imazalil, triadimenol, difenoconazole, myclobutanil, prothioconazole, triticonazole and tebuconazole. Example of morpholines include but are not limited to dimethomorph, fenpropidine and fenpropimorph. Examples of strobilurins include but are not limited to azoxystrobin, kresoxim-methyl and analogues. Examples of phthalonitriles include but are not limited to chlorothalonil, mancozeb, and fluazinam.

Examples of the suitable herbicides include but are not limited to allidochlor, beflubutamid, benzadox, benzipram, bromobutide, cafenstrole, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid, tebutam, chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen propanil, benzoylprop, flampropand flamprop-M; acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor, xylachlor; benzofluor, perfluidone, pyrimisulfan, asulam, carbasulam, fenasulam oryzalin, bilanafos; chloramben, dicamba, tricamba, bispyribac, pyriminobac, pyritbiobac, chlorthal, aminopyralid, clopyralid and picloram; quinclorac and quinmerac, cacodylic acid, hexaflurate, potassium arsenite, sodium arsenite, mesotrione, sulcotrione, tefuryltrione, tembotrione, benfuresate and ethofumesate; asulam, carboxazole chlorprocarb, dichlormate, fenasulam, karbutilate, barban, BCPC, carbasulam, carbetamide, CEPC, chlorbufam, chlorpropbam, CPPC, desmedipham, phenisopham, phenmedipham, phenmedipham-ethyl, propham, swep, alloxydim, butroxydim, clethodim, cloproxydim, cycloxydim, profoxydim, sethoxydim, tepraloxydim, tralkoxydim; isoxachlortole, isoxaflutole, benzfendizone, cinidon-ethyl, flumezin, flumiclorac, flumioxazin, flumipropyn; benfluralin, butralin, dinitramine, etbalfluralin, fluchloralin, isopropalin, methalpropalin, nitralin, oryzalin, pendimethalin, prodiamine, profluralin, trifluralin; dinofenate, dinoprop, dinosam, dinoseb, dinoterb, DNOC, etinofen, acifluorfen, aclonifen, bifenox, chlomethoxyfen, chlormitrofen, etnipromid, fluorodifen, fluoroglycofen, fluoronitrofen, fomesafen, furyloxyfen, halosafen, lactofen, nitrofen, nitrofluorfen, oxyfluorfen, dazomet, alorac, chloropon, dalapon, flupropanate, hexachloroacetone, iodomethane, methyl bromide, monochloroacetic acid, imazamethabenz, imazamox, imazapic, imazapyr, imazaquin and imazethapyr, bromobonil, bromoxynil, chloroxynil, dichlobenil, jodobonil, ioxynil and pyraclonil, amiprofos-methyl, anilofos, bensulide, bilanafos, butamifos, fosamine, glufosinate, glyphosate, piperophos, bromofenoxim, clomeprop, difenopenten, etnipromid, fenteracol and trifopsime, fenoprop, mecopropand mecoprop-P; chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; dinitramine and prodiamine, benzofenap, pyrazolynate, pyrasulfotole, pyrazoxyfen, pyroxasulfone and topramezone.

Examples of nematicides include but are not limited to abamectin, benomyl, carbofuran, carbosulfan, cleothocard; alanycarb, aldicarb, aldoxycarb, oxamyl, diamidafos, fenamiphos, fosthietan, phosphamidon, cadusafos, chlorpyrifos, dichlofenthion, dimethoate, ethoprophos, fensulfothion, fosthiazate, heterophos, isamidofos, isazofos, methomyl, phorate, phosphocarb, terbufos, thiodicarb, thionazin, triazophos, imicyafos, mecarphon, acetoprole, benclothiaz, chloropicrin, dazomet, 1,2-dichloropropane, 1,3-dichloropropene, furfural, iodomethane, metam, methyl bromide, methyl isothiocyanate, and xylenols.

The invention will now be described with respect to the following examples which do not limit the disclosed method in any way and only exemplify the claimed method. It will be apparent to those skilled in the art that various modifications and variations can be made to the method/process of the present disclosure without departing from the scope of the disclosure. Other embodiments will be apparent to those skilled in the art from consideration of the specification and practice of the method/process disclosed herein. It is intended that the specification and examples be considered as exemplary only, with a true scope of the disclosure being indicated by the following claims and their equivalent.

### EXAMPLES

### Example 1: Preparation of Encapsulated Composition Comprising of Extracts of Essential Oils in Accordance with an Exemplary Embodiment

Composition: The composition of extracts of essential oils used in the exemplary composition of the present disclosure and their quantity are listed in Table 1, below:

TABLE 1
Composition of extracts of essential
oils in encapsulated composition

| S. | | Quantity |
|---|---|---|
| No. | Component | (in w/w %) |
| 1. | Eugenol | 50 |
| 2. | Cinnamaldehyde | 20 |
| 3. | D-limonene | 15 |
| 4. | Linalool | 5 |
| 5. | Beta-caryophyllene | 5 |
| 6. | Cardanol acetate | 5 |

Purification of clay: 100 grams of bentonite clay was taken and heated in a hot air oven at 250° C. for 4 hours. To the heated bentonite clay, 500 ml of distilled water was added with stirring at 2000 rpm for 30 minutes to obtain a suspension of the bentonite clay. The obtained suspension of bentonite clay was allowed to separate into a sediment of bentonite clay and an aqueous layer. Post separation, the aqueous layer was decanted, and 50 ml of hydrochloric acid was added to the sediment of bentonite clay at 27° C. with stirring at 2500 rpm for 60 minutes. The obtained sediment of clay was dried in the hot air oven at 200° C. for 6 hours to obtain purified bentonite clay.

Preparation of encapsulated composition: To 100 grams of the purified clay, 250 ml of distilled water was added to prepare an aqueous slurry of clay. This aqueous slurry was stirred for 60 minutes at a stirring rate of 3500 rpm, followed by addition of 20-30 ml of sodium hydroxide till the pH of the aqueous slurry of the clay is 10. After maintaining the pH of the aqueous slurry of the clay to the desired value, a mixture of the extract of essential oils as mentioned above in table 1 was added dropwise to the aqueous slurry dropwise at a rate of 25 μl/sec. After the completion of the addition of mixture of the extract of essential oils to the aqueous slurry of the clay, the mixture of the slurry was stirred at 4000 rpm for 20 minutes. The mixture of the slurry was poured in stainless-steel trays and dried in the hot air oven with an air circulation at 50° C. to obtain encapsulated composition comprising the extract of essential oils microencapsulated with clay.

### Comparative Example 1

(i) Preparation of essential oil composition EO_CD using cyclodextrin as an encapsulation material:
Preparation of cyclodextrin solution: 50 grams of β-cyclodextrin was taken and dissolved in 500 ml of distilled water to prepare β-cyclodextrin solution. The solution was heated at 50° C. until the β-cyclodextrin was completely dissolved in water.
Preparation of the essential oil composition: To the prepared β-cyclodextrin solution, 5 ml of mixture of the extract of essential oils as mentioned above in table 1 was added dropwise at a rate of 25 μl/sec under continuous stirring. After the completion of the addition of mixture of the extract of essential oils to the β-cyclodextrin solution, the obtained mixture was stirred at a stirring rate of 2500 rpm for 20 minutes. Post stirring, 500 ml of ethanol was added to the mixture, followed by the stirring of the mixture at 50° C. for 2 hours. The mixture was allowed to cool at room temperature to allow the formation of precipitate. The cooled mixture was filtered to separate the formed precipitate from the filtrate. The obtained filtrate was air-dried or freeze dried to obtain the essential oil composition comprising the extract of essential oils microencapsulated with the β-cyclodextrin.

(ii) Preparation of essential oil composition EO_PVA using polyvinyl alcohol (PVA) as an encapsulation material:
Preparation of PVA solution: 10 grams of PVA was taken and dissolved in 1 litre of distilled water to prepare PVA solution. The solution was heated at 85° C. under continuous stirring, until the PVA is completely dissolved in water. The obtained solution was allowed to cool at room temperature under constant stirring to avoid the formation of a film on the surface.
Preparation of the essential oil composition: To the prepared PVA solution, 10 ml of mixture of the extract of essential oils as mentioned above in table 1 was added dropwise at a rate of 25 μl/sec. After the completion of the addition of mixture of the extract of essential oils to the PVA solution, the obtained mixture was homogenized using an emulsifier or high shear mixer at 5000 rpm to form a stable oil in water emulsion. The obtained emulsion was poured into a mould to form a film. The formed film was freeze-dried, followed by standard lyophilization procedures to obtain the dried product. The obtained dried product was grinded using pulveriser to obtain the essential oil composition comprising the extract of essential oils microencapsulated with the β-cyclodextrin.

(iii) Preparation of essential oil composition EO_CT using chitosan as an encapsulation material:
Preparation of chitosan solution: 2 grams of chitosan was taken and dissolved in 100 ml of 1% acetic acid solution. The solution was stirred at room temperature to completely dissolve the chitosan in the acetic acid solution.
Preparation of the essential oil solution: The mixture of the extract of essential oils as mentioned in table 1 was taken and mixed with 50 ml of ethanol to prepare an essential oil solution.
Preparation of the essential oil composition: To the prepared chitosan solution, 10 ml of the essential oil solution prepared above was added dropwise at a rate of 25 μl/sec to form a mixture. After the completion of the addition of the essential oil solution to the chitosan solution, the obtained mixture was stirred at a stirring rate of 3000 rpm for 30 minutes. The obtained mixture was subjected to ultra-sonication (sonics, 750 W) assisted homogenization. Ultrasonication was performed under the following conditions-Pulse on-5 seconds, pulse-off-5 seconds for 30 minutes to form a stable emulsion.
After the preparation of emulsion, 0.5 grams of sodium tripolyphosphate was taken and dissolved in 100 ml of water to prepare 0.5% solution of sodium tripolyphosphate. The solution of sodium tripolyphosphate was added dropwise to the prepared emulsion under continuous stirring at a stirring rate of 4000 rpm for 2 hours to form microspheres of the essential oil composition comprising the extract of essential oils microencapsulated with chitosan. The obtained microspheres were separated from the solution by centrifugation, followed by washing with distilled water to remove any unreacted material. The dried microspheres of the essential oil composition comprising the extract of essential oils microencapsulated with chitosan is freeze dried to remove any remaining water.

The percentage release of extract of essential oils from EO1, EO_CD, EO_PVA and EO_CT was compared over a period of 90 days.

Results and Observation: It was found that the essential oil compositions EO_CD, EO_PVA and EO_CT prepared using conventional encapsulating materials-cyclodextrin, PVA, and chitosan, respectively, did not show sustained release of the essential oils for a prolonged period of time as compared to the essential oil composition EO1 prepared using silane modified clay.

### Example 2: Preparation of Encapsulated Composition Comprising of Urea in Accordance with an Exemplary Embodiment

Purification of clay: 100 grams of bentonite clay was taken and heated in a hot air oven at 250° C. for 4 hours. To the heated bentonite clay, 500 ml of distilled water was added with stirring at 2000 rpm for 30 minutes to obtain a suspension of the bentonite clay. The obtained suspension of bentonite clay was allowed to separate into a sediment of bentonite clay and an aqueous layer. Post separation, the aqueous layer was decanted, and 50 ml of hydrochloric acid was added to the sediment of bentonite clay at 27° C. with stirring at 2500 rpm for 60 minutes. The obtained sediment of clay was dried in the hot air oven at 200° C. for 6 hours to obtain purified bentonite clay.

Preparation of encapsulated composition: To 100 grams of the purified clay, 250 ml of distilled water was added to prepare an aqueous slurry of clay. The slurry was stirred at 4000 rpm for 30 minutes. To the prepared aqueous slurry of clay, 50 grams of urea fertilizer granules and 5 ml of castor oil were added and mixed thoroughly. The granules were mixed uniformly to ensure that granules are uniformly coated with the aqueous slurry of clay. The mixing of the aqueous slurry of clay and urea granules can also be done in a rotary drum to ensure uniform coating of the urea granules by clay.

The thickness of coating of the aqueous slurry of the clay on the urea granules depends on the amount of time the granules are left in the aqueous slurry of the clay. The obtained coated urea granules were spread in stainless-steel trays and dried in the hot air oven with an air circulation at 35° C. to obtain encapsulated composition comprising the urea pellets microencapsulated with clay.

### Example 3: Preparation of Encapsulated Composition Comprising Seaweed in Accordance with an Exemplary Embodiment

Purification of clay: 100 grams of bentonite clay was taken and heated in a hot air oven at 250° C. for 4 hours. To the heated bentonite clay, 500 ml of distilled water was added with stirring at 2000 rpm for 30 minutes to obtain a suspension of the bentonite clay. The obtained suspension of bentonite clay was allowed to separate into a sediment of bentonite clay and an aqueous layer. Post separation, the aqueous layer was decanted, and 50 ml of hydrochloric acid was added to the sediment of bentonite clay at 27° C. with stirring at 2500 rpm for 60 minutes. The obtained sediment of clay was dried in the hot air oven at 200° C. for 6 hours to obtain purified bentonite clay.

Preparation of the seaweed extract: 100 grams of seaweed extract was taken and mixed with 500 ml of water to prepare a seaweed solution. The obtained solution was heated at 90° C. for 2 hours, followed by cooling at room temperature. The solution was allowed to separate into the supernatant and the precipitate. Post separation, the obtained supernatant was concentrated by boiling at 100° C. till 100 ml of supernatant was left. The pH of the obtained supernatant was adjusted to 10 by adding NaOH to obtain the seaweed extract.

Preparation of encapsulated composition: To 100 grams of the purified clay, 250 ml of distilled water was added to prepare an aqueous slurry of clay. This slurry was stirred at 4000 rpm for 30 minutes. To the prepared aqueous slurry of clay, 10 ml of the prepared seaweed extract was added slowly under continuous stirring to form a homogenous mixture. In the prepared homogenous mixture, 10 grams of sodium alginate was added, followed by stirring at a stirring rate of 2500 rpm at 80° C. to form a solution. Post stirring, 1% of calcium chloride solution was added to the solution to form a gel like structure. The obtained gel like solution was dried using a spray dryer to obtain encapsulated composition comprising seaweed microencapsulated with clay.

### Example 4: Preparation of Encapsulated Composition Comprising of Live Bacterial Cells in Accordance with an Exemplary Embodiment

Purification of clay: 100 grams of bentonite clay was taken and heated in a hot air oven at 250° C. for 4 hours. To the heated bentonite clay, 250 ml of distilled water was added with stirring at 4000 rpm for 30 minutes to obtain a suspension of the bentonite clay. The obtained suspension of bentonite clay was allowed to separate into a sediment of bentonite clay and an aqueous layer. Post separation, the aqueous layer was decanted, and 50 ml of hydrochloric acid was added to the sediment of bentonite clay at 27° C. with stirring at 3500 rpm for 60 minutes. The obtained sediment of clay was dried in the hot air oven at 200° C. for 6 hours to obtain purified bentonite clay.

Preparation of encapsulated composition: To 100 grams of the purified clay, 250 ml of distilled water was added to prepare an aqueous slurry of clay. This aqueous slurry was stirred for 60 minutes at a stirring rate of 3500 rpm, followed by addition of 20-30 ml of sodium hydroxide till the pH of the aqueous slurry of the clay is 10. After maintaining the pH of the aqueous slurry of the clay to the desired value, 100 ml of bacterial suspension (109 cells/ml) was added drop by drop at 10-25 μl/sec. After the completion of the addition of bacterial suspension to the aqueous slurry of the clay, the mixture of the slurry was stirred at 2000 rpm for 30 minutes. The mixture of the slurry was poured in stainless-steel trays and dried in the hot air oven with an air circulation at 50° C. to obtain encapsulated composition comprising the bacterial cells microencapsulated with clay.

Preparation of sample for assessment of heat resistance of encapsulated composition: 1 gm of encapsulated bacterial material was mixed with 10 ml of sterile water in a test tube. Similarly. 1 gm of bacterial suspension (without encapsulation) was mixed with 10 ml of sterile water in another test tube. Both the test tubes were heated at 150° C. in a water bath for 1 hour. After one hour, both the test tubes were kept at room temperature. Next, 100 μl of control bacterial suspension and the two heated suspensions were spread on the nutrient agar plates, followed by incubation for 24 hours. The plates were evaluated for colony count.

Results and Observation: The colony count for each of the samples stated above are listed in table 3 below:

TABLE 3
Colony count of various samples of
bacterial suspension

| S. No. | Treatment | CFU/plate |
|---|---|---|
| 1 | Control bacterial suspension | 4128 ± 80 |
| | (without heating and | |
| | encapsulation) | |
| 2. | Heated, encapsulated | 3418 ± 164 |
| | bacterial suspension | |
| 3. | Non-encapsulated, heated | 352 ± 13 |
| | bacterial suspension | |

It was observed that encapsulation in accordance with the present disclosure substantially improved the heat resistance of the bacterial suspension.

### INDUSTRIAL APPLICATION

The disclosed encapsulated composition finds application in the food, agricultural/environmental, cosmetic, and pharmaceutical industries. Specifically, the disclosed encapsulated composition finds application as a carrier for use in food packaging and preservation compositions. In the agricultural area, it allows for reducing losses by providing better biological integrity, increasing efficiency, and increasing formulation stability. The disclosed encapsulation composition has been found to be efficient for the formulation of sustained-release pesticides. The disclosed composition exhibits controlled release of the active ingredient while causing no or negligible degradation of the active ingredient.

The disclosed encapsulated composition improves the stability of formulated systems, increases the release time, reduces lipid oxidation, and protects active properties even in extreme thermal and pH conditions. Also, the disclosed composition increases the bioavailability of the active ingredient and protects it against hostile conditions, such as high temperatures and the presence of oxygen and light.

We claim:

1. A process for preparing an encapsulated composition, the process comprising the steps of:
a. heating a clay at a temperature in a range of 200 to 250° C. for a time period in a range of 3-5 hours;
b. mixing the heated clay with water in a w/w ratio in a range of 1:5-1:10 to obtain a suspension of clay, followed by separation of suspension of clay into a sediment of clay and an aqueous layer;
c. decanting the aqueous layer, followed by the addition of an acid to the sediment of clay to obtain purified clay;
d. preparing an aqueous slurry of the purified clay;
e. adjusting the pH of the aqueous slurry of the purified clay in the range of 9-11;
f. blending at least one active ingredient with the aqueous slurry of the purified clay to obtain a mixture of the active ingredient and the purified clay; and
g. subjecting the mixture of the active ingredient and the purified clay to drying such that the encapsulated composition comprising of the active ingredient homogenised with the clay is obtained.

2. The process of claim 1, wherein the active ingredient is selected from the group consisting of essential oils or extracts thereof, bio-stimulants, fertilizers, pesticides and any blend of natural ingredients.

3. The process of claim 1, wherein the active ingredient is mixed with a solvent selected from the group consisting of ethanol, isopropanol, and butanol, prior to blending of the active ingredient with the aqueous slurry of the purified clay.

4. The process of claim 1, wherein the active ingredient is blended with the aqueous slurry of the purified clay in a w/w ratio in the range of 1:5-2:5.

5. The process of claim 4, wherein the acid is hydrochloric acid having a molarity in the range of 3-7 M.

6. The process of claim 1, wherein the base is selected from the group consisting of sodium hydroxide, potassium hydroxide, calcium hydroxide and combinations thereof.

7. The process of claim 1, wherein the clay is selected from the group consisting of montmorillonite (MMT), bentonite clay, kaolinite, dickite, amesite, lizardite, smectite, palygorskite, sepiolite, glauconite, mica, vermiculite, saponite halloysite, hectorite and combinations thereof.

8. The process of claim 7, wherein the clay is bentonite clay selected from the group consisting of sodium bentonite, calcium bentonite and combinations thereof.

9. The process of claim 8, wherein the bentonite clay has an average particle size in the range of 1-10 μm, a mass density in the range of 1.2-1.8 g/cm³, and a cation exchange capacity in the range of 60 to 150 meq/100 g.

10. The process of claim 1, wherein the drying is carried out till the moisture of the mixture of the active ingredient and the purified clay is less than 5-8% as detected by loss in weight method at 120° C.

11. An encapsulated composition comprising at least one active ingredient homogenised with a clay wherein the active ingredient is selected from the group consisting of essential oils, bio-stimulants, fertilizers, pesticides, and any blend of natural ingredients.

12. The encapsulated composition of claim 11, comprising the active ingredient and the clay in a w/w ratio ranging between 0.5:100 to 5:100.

13. The encapsulated composition of claim 11, wherein the clay is selected from the group consisting of montmorillonite (MMT), bentonite clay, kaolinite, dickite, amesite, lizardite, smectite, palygorskite, sepiolite, glauconite, mica, vermiculite, saponite halloysite, hectorite, and combinations thereof.

14. The encapsulated composition of claim 13, wherein the clay is bentonite clay selected from the group consisting of sodium bentonite, calcium bentonite, and combinations thereof.

15. The encapsulated composition as claimed in claim 11, wherein the composition is in the form of microspheres having an average particle size (D50) in the range of 0.5 to 5 μm.
